(19) United States
(12) Patent Application Publication
(10) Pub. No.: US 2006/0025722 A1
(43) Pub. Date: Feb. 2, 2006

(54) Continuous nasal irrigation device

(76) Inventor: John Andrew Turner, Friendswood, TX (US)

Correspondence Address: John Andrew Turner, 714 Stonecreek Circle, Friendswood, TX, 77546, US

(21) Appl. No.: 10/903,539

(22) Filed: Jul. 30, 2004

(51) Int. Cl. A61M 5/32

(52) U.S. Cl. 604/180; 604/275

(57) ABSTRACT

A generally flat, specially-shaped plastic bag which holds a sterile fluid, such as water, saline, or medication. The shape of the bag symmetrically follows the contours of the upper face and connects across the bridge of the nose. Each cheek section ends in a nostril section comprising a small tube plugged by a flow-adjustable wick which is positioned into its adjacent nostril. The apparatus is held in place on the face of the patient by an adhesive means, such as two-sided adhesive tape. To use the device, a patient removes the removable caps, exposes the adhesive means, and applies it to the face, inserting each wick into its adjacent nostril, allowing fluid to slowly seep into the nostrils over several hours, while either asleep or awake. This thereby continually promotes the clearing of thick, overabundant, irritating mucous created by diseases like colds, sinusitis, or allergic rhinitis.

### FIELD OF THE INVENTION

The present invention relates to nasal irrigation and, more particularly, to providing continuous moisturization to the nasal membranes of a human patient, or wearer, over a long period of time by a small wearable device.

### BACKGROUND OF THE INVENTION

Upper respiratory diseases, including viral upper respiratory infections (the common cold), sinusitis, pharyngitis, laryngitis, bronchitis, and allergic and non-allergic rhinitis account for significant morbidity, days lost from work, and millions if not billions of dollars spent annually for medical treatment and symptom relief. It is estimated that children have on average 6-8 viral upper respiratory infections annually, while adults average 2-4.

The internal lining of the nose and sinuses is a mucosa and has several different functions, which include filtering, humidifying, and warming the inspired air, aided in part by producing a thin layer of mucous. As a result of infectious illnesses, or allergic or nonallergic rhinitis, the nasal mucosa reacts by swelling and hypersecreting mucous. This excess mucous is irritating to tissues it contacts, which can lead to comoroid conditions, such as sinusitis, laryngitis, pharyngitis, bronchitis, serous otitis, and even gastritis. This overabundant mucous tends to accumulate and respiration makes it become more dry and viscous, thereby becoming more difficult to clear. This leads to clogging and inflamation of adjacent anatomic structures, causing worsening of symptoms and an increased likelihood of further complications.

During waking hours, typically most time is spent sitting or standing. In this upright position, gravity, assisted by reflexive sniffing and swallowing, helps to clear this excess mucous to the esophagus and the stomach. During sleeping hours, however, no significant help in clearing mucous occurs from gravity, sniffing, or swallowing. The mucous hypersecretion as well as viscosity-increasing respiration continues unabated, so the upper respiratory tissues become especially irritated from the more profuse accumulation of hyperviscous mucous during sleep. Hydrating the thick, viscous mucous makes it less viscous, more easily cleared, and thereby reduces the symptoms and comorbitities as discussed.

Other solutions in existence include the following:
1. Methods to hydrate the inflamed nasal mucosa for symptom relief includes anything which brings water to the nose. Externally, this can be accomplished by breathing humidified air or steam, taking a shower, swimming, or using nasal saline spray.
2. Adequate total body hydration status helps to keep the overabundant mucous less viscous, minimizing mechanical obstruction.
3. Several devices were found which aid in bringing water or saline to the nasal mucosa.
4. One device which helps to keep the nasal passages patent is the Breathe Right (R) nasal strip.
5. Sleeping in a semi-upright position, such as in a chair, so that gravity will continue to help clear the mucous.

Shortcomings of the other solutions include the following:
1. Hydrating fluids like saline or water definitely help to clear the thick mucous, but their effects are relatively short-lived as mucous continues to be produced and breathing continues to dry it out.
2. Optimizing total body hydration status helps, too, but by itself not enough to adequately decrease the mucous viscosity.
3. The available devices for saline irrigation tend to be too complex, expensive, and cumbersome for routine home use.
4. The Breathe Right (R) nasal strip helps promote patent nasal airways, is simple to use, affordable, and helps symptoms, but does not directly address mucous hypersecretion and its associated problems. Because it improves the airflow over the abundant mucous, the nasal strip may cause it to become more viscous than it otherwise would be. So it offers tangible oat suboptimal relief from symptoms associated with diseases which cause nasal mucous hypersecretion.
5. Sleeping upright in a chair, which uses assistance from gravity, can help with mucous clearing to a degree, and likely helps to ensure that more of the cleared mucous flows to the esophagus and stomach rather than into the trachea and bronchi. Poor sleep quality, neck, and back strains are common when this remedy is employed. It is only of marginal help in most cases.

It is therefore an object of the invention to provide an inexpensive way to moisturize nasal membranes of a patient, including while he or she sleeps.

It is another object of the invention to minimize respiratory symptoms of diseases like colds, sinusitis, and allergic rhinitis.

It is another object of the invention to make thick, irritating mucous in the upper respiratory tract to become less viscous and tenacious, and to aid in its clearing, thereby making the upper respiratory tract less inflamed, less uncomfortable, and less obstructed.

It is another object of the invention to hasten recovery from symptoms of the diseases of the upper airway while minimizing the risk of complications, including bacterial superinfection, by helping to clear the mucous in these diseases.

It is another object of the invention to provide a potential means for delivering medication to the nasal membranes over a prolonged period of time.

### SUMMARY OF THE INVENTION

In accordance with the present invention, there is provided a generally flat, specially-shaped plastic bag which holds fluid, such as sterile saline or water. The shape of this bag follows the contours of the upper face of a patient and has two cheek sections, which are symmetric and connect through a narrow but open section across the bridge of the nose. The upper portion of each cheek section follows a path below the lower eyelid margin. The side spans inferiorly from below each eyelid well in front of the ear to a level slightly below the bottom of the nose. The bottom of each cheek section traverses the lower cheek to the bottom of the nose, where it ends in a tube which turns upward into the ipsilateral nostril. This tube ends in a wick which may be optionally adjustable to regulate the flow rate of the fluid.

The device is applied over the nose and cheeks by removing the cap from each nostril section, peeling off the protective covering of the adhesive tape on the back side of it, and applying it appropriately to the face while placing a wick into each nostril of the patient. Through capillary action, the fluid is then allowed to slowly seep into each nostril over several hours, even during sleep, to help to control the thick, overabundant mucous present in diseases like colds, sinusitis, or allergic rhinitis, thereby keeping the nose and other airways more open and less uncomfortable and minimizing the symptoms of these common ailments. The potential exists for this fluid to be used as a vehicle through the use of this device for delivering medication to the nasal mucosa as well.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of a device in accordance with the present invention being worn by a patient;

FIG. 2 is a front view of a device shown in FIG. 1;

FIG. 3 is a rear view of an inventive device;

FIG. 4 is a rear view or an alternative embodiment for the two-sided adhesive tape;

FIG. 5 is a cross-sectional view of a device; and

FIG. 6 is a lower frontal view of a device with the removable cap in place.

For purposes of clarity and brevity, like elements and components will bear the same designations and numbering throughout the FIGURES.

### DESCRIPTION OF THE PREFERRED EMBODIMENT

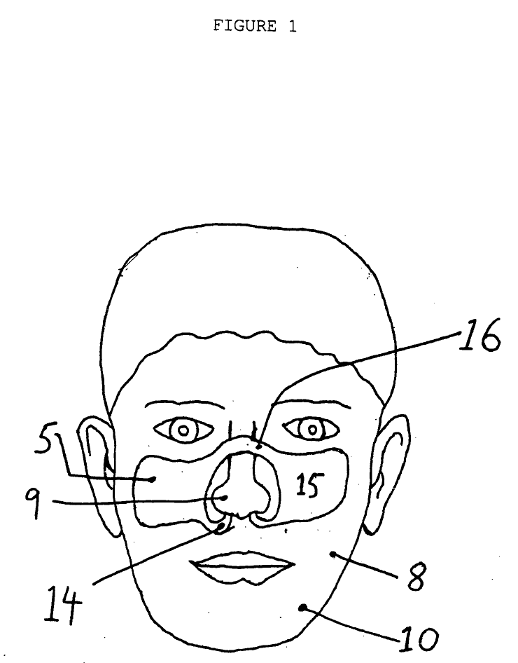

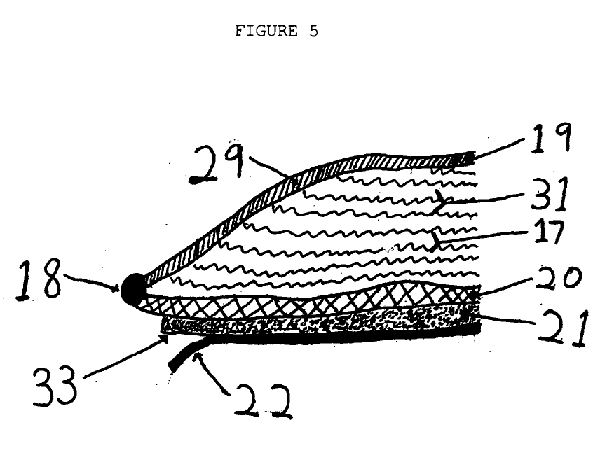

A nasal irrigation device 5 in accordance with the present invention is illustrated in situ in FIG. 1. Of note is the symmetrical positioning on the face 8 of a patient 10, which is defined as a wearer of said device 5. A cheek section 15 overlies each cheek and acts as a reservoir for fluid 17 (FIG. 5). Each cheek section 15 is lumenally continuous with one another through a nose bridge section 16. Each cheek section 15 also projects distally and medially to form a nostril section 14, which as its name implies, inserts into the distal part of the ipsilateral nostril 9 when a device 5 is being worn by a patient 10.

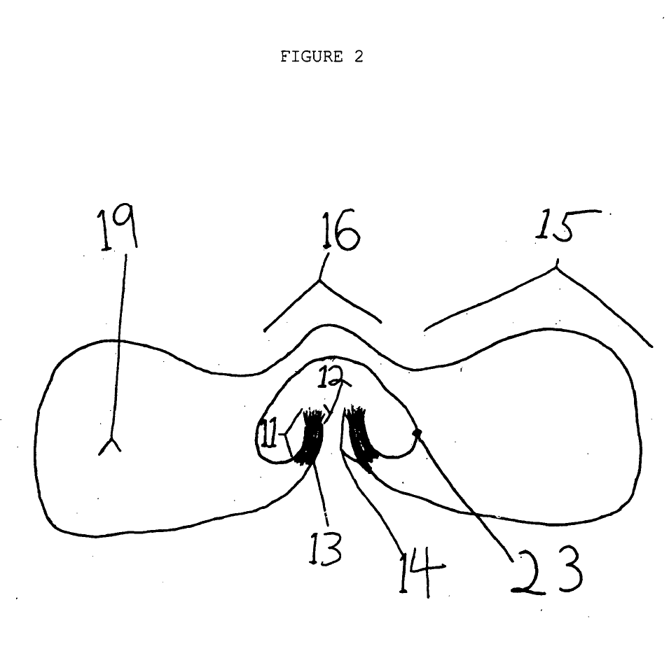

A frontal view of a device 5 is shown in FIG. 2. Each nostril section 14 includes a wick 11. The retained wick 13 portion is roughly cylindrical in shape and is bordered and frictionally retained by a front flexible plastic wall 19, a back flexible plastic wall 20 (FIG. 5) and their connecting heat welded watertight outer seam 18 and inner seam 23. An exposed wick 12 portion is the remaining portion of said wick 11. When said device 5 is being worn, said exposed wick 12 is in contact with the nasal mucosa (not shown) of each nostril 9 and is the site of fluid 17 transfer from a device 5 to the nasal mucosa of a patient 10. The materials used for creating said front flexible plastic wall 19 and back flexible plastic wall 20 as well as the heat welding methods to create the watertight inner seam 23 and outer seam 18 can be inexpensive and similar to those used in manufacturing Zip Lock (R) bags.

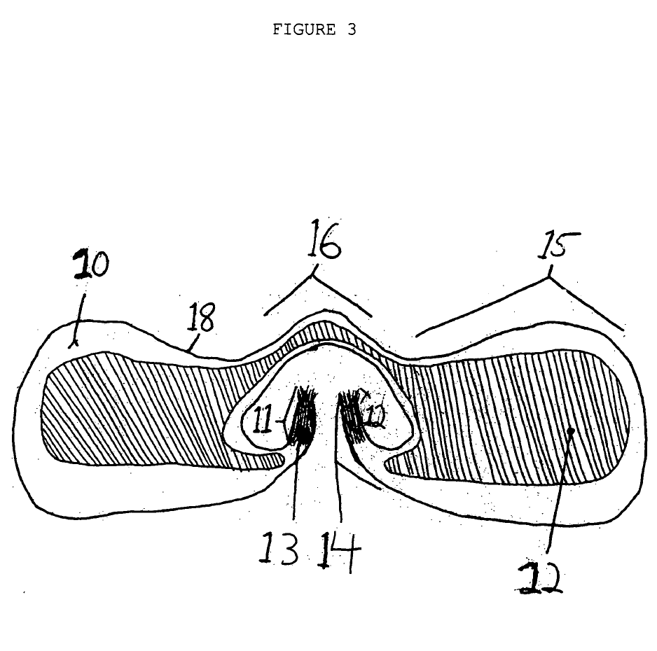

FIG. 3 shows the back view of a device 5. This view is essentially the same as the frontal view (FIG. 1) except that the adhesive means 33, such as two-sided adhesive tape 21 with a peelable adhesive cover 22 (FIGS. 3, 4, and 5), has been placed on said back flexible plastic wall 20 during the manufacturing process. One surface of said two-sided adhesive tape 21 adheres to said back flexible plastic wall 20 and a peelable adhesive cover 22 is provided to cover the other adhesive surface of said two-sided adhesive tape 21. When said peelable adhesive cover 22 is removed, the adhesive portion of said two-sided adhesive tape 21 is exposed and is used to adhere said device 5 to the face 8 of a patient 10 with each nostril section 14 positioned in its corresponding nostril 9. Of note is the distribution of adhesive means 33 covered by a peelable adhesive cover 22 over most of each cheek section 15. The inferior part of each cheek section 15 is left uncovered so that if the patient 10 has a beard, the adhesive means 33 will be less likely to adhere uncomfortably to his hair.

From each cheek section 15, the adhesive means 33, such as two-sided adhesive tape 21, is continuous across a nose bridge section 16 and also extends to the most proximal part of each nostril section 14. This configuration allows a patient 10 to remove the entire peelable adhesive cover 22 with one movement prior to positioning. A patient 10 must take care not to allow the exposed adhesive surface of said adhesive means 33 to contact anything other its intended mounting surface, as that might render said device 5 not usable.

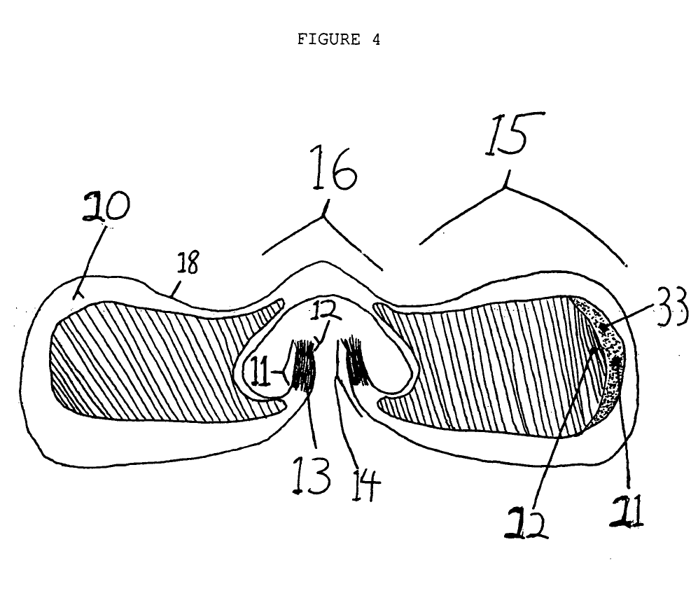

An alternative embodiment, as seen in FIG. 4, provides the adhesive means 33, such as two-sided adhesive tape 21, in two separate areas on the upper part of each cheek section 15 on said back flexible plastic wall 20. Unlike the first embodiment shown in FIG. 3, however, there is no continuation of said adhesive means 33 across the nose bridge section 16. The patient 10 applies each side of a device 5 sequentially in this embodiment. The advantages of this configuration is that it is less likely that a patient 10 will accidentally adhere the exposed adhesive means 33 to surfaces other than the intended mounting surface of his or her face 8 since there is less exposed surface area of said adhesive means 33. Since there is no adhesion of a device 5 across the bridge of the nose, however, said device 5 may be more unstable when mounted. Moreover, a separate peelable adhesive cover 22 over each cheek section 15 must be peeled for proper device 5 mounting.

FIG. 5 shows a cross-sectional view of a device 5 at the lateral part of a cheek section 15 to further illustrate the components and their positional relationships to one another. Of note in this figure is a front flexible plastic wall 19 and its attachment to a back flexible plastic wall 20 by an outer seam 18. The flexible contoured watertight structure comprising said front flexible plastic wall 19 and said back flexible plastic wall 20 as well as the connecting inner seam 23 and outer seam 18 creates the outer boundary of a cavity 31 containing fluid 17, and is referred to as a bag 29. Said cavity 31 is defined as the potential space within the confines of said bag 29 in which a fluid 17 is contained. Said fluid 17 can potentially be a number of sterile liquid formulations, including a sterile saline solution, such as a buffered physiological isotonic saline, a hypertonic saline solution, water, or a solution containing other ingredients or concentrations, including medications, intended to be delivered to the nasal mucosa of a patient 10. Using said fluid 17 as a vehicle, said device 5 may thus act as a means to deliver moisture and potentially medication the the nasal mucosa and the rest of the upper respiratory tract of a patient 10.

The only outlets for a fluid 17 from said bag 29 are through either nostril section 14 and its corresponding flow-regulating wick 11. Said cavity 31, as a potential space for a fluid 17, will preferably not be filled to its capacity. If the walls of said bag 29 are under tension from its cavity 31 being completely full, then the hydraulic pressure within this cavity 31 lumen will be elevated even more if a patient 10 lies on one cheek. This increase in pressure would cause a detrimental increase in the flow rate of said fluid 17 out of said device 5 to one greater than that which would be attained by capillary action alone. If part of the potential volume in said cavity 31 was left unfilled, then a smaller increase in the pressure of said fluid 17 will occur when external pressure is applied to one cheek section 15, as some fluid 17 will shift across said nose bridge section 16 to the contralateral cheek section 15 instead of increasing the pressure within said cavity 31 while leaving each wick 11 in contact with fluid 17 for continued flow. A minimal, if any, amount of air or other gases would be present with said fluid 17 in the cavity 31, as gas would occupy volume and displace fluid 17 within said cavity 31, and gas could disrupt capillary action, and thereby fluid 17 delivery, from said device 5 to the nasal mucosa of a patient 10.

Also shown in FIG. 5 is the adhesive means 33, which most practically would be two-sided adhesive tape 21 such as the type used in Breathe Right (R) nasal strips. Said adhesive means 33 is attached to said back flexible plastic wall 20 during the manufacturing of a device 5 leaving said peelable adhesive cover 22 in place for a patient 10 to remove, as described hereinabove.

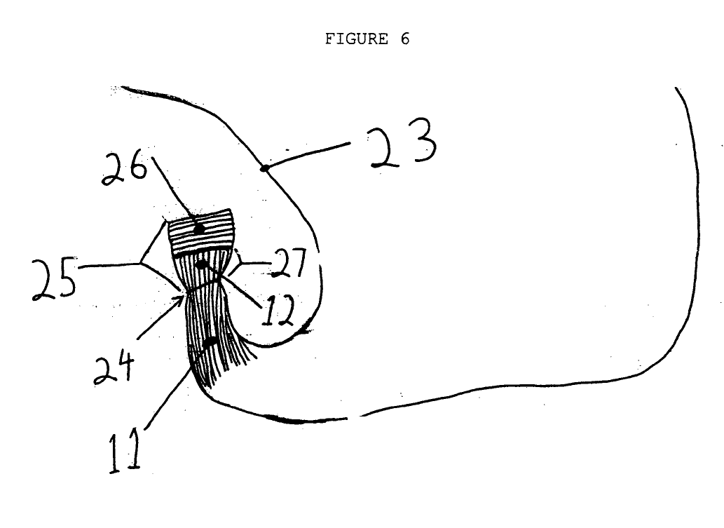

Since leakage of fluid 17 through a nostril section 14 prior to use is undesirable, some means of fluid 17 containment is needed. FIG. 6 illustrates the preferred means, which is a removable cap 25 at the distal part of each nostril section 14 that covers said exposed wick 12 using a continuation of said front flexible plastic wall 19 and back flexible plastic wall 20. Said removable cap 25 ideally has a textured grip 26 segment at its most distal area, which is textured with ribs or other grip-improving texture. Said textured grip 26 is isolated from a cap body 27, which is between said textured grip 26 and said cap neck 24, such that none of the strands of said exposed wick 12 or said fluid 17 extends past said cap body 27 to said textured grip 26. Grasping and pulling said removable cap 25 off of its nostril section 14 by said textured grip 26 therefore does not also pull said wick 11 from its nostril section 14, as this would render said device 5 not useable. Said removable cap 25 is removed from the tip of each nostril section 14 by said patient 10 at time of desired use of a device 5 by grasping a nostril section 14 with the thumbnail at Its cap neck 24. Then, with the other hand said patient 10 grasps its textured grip 26 and pulls said removable cap 25 away from its nostril section 14. The pressure applied by the thumbnail at said cap neck 24 also prevents said wick 11 from becoming dislodged and helps to assure that the shear force is favorable for a tear to occur at said cap neck 24. To further optimize the proper tear line when a removable cap 25 is removed, its cap neck 24 may be made of thinner plastic along its narrow segment.

Operation of said device 5 is relatively straightforward. When use is desired, each removable cap 25 is removed as outlined previously, followed by removal of said peelable adhesive cover 22 or covers. Said device 5 is then carefully positioned into place as illustrated in FIG. 1 and adhered to the face 8 using gentle pressure over those parts of said device 5 which have said adhesive means 33, such as two-sided adhesive tape 21. The distal part of each nostril section 14 is inserted gently into each nostril 9, and said device 5 is then installed and functioning. Said fluid 17 is slowly dispersed to the nasal mucosa typically over a period of hours, and can be used while a patient 10 is awake or asleep. Prior to application, said exposed wick 12 may be trimmed to a shorter length using scissors if a slower rate of flow is desired, or said exposed wick 12 may be pulled slightly from its nostril section 14 to make it longer if a faster fluid 17 flow rate is desired. Said adhesive means 33 should not be applied to skin which is irritated or infected, and if any skin irritation should-develop after applying a device 5, its use should be discontinued. A patient 10 with particularly oily facial skin may achieve better adhesion of a device 5 if the oils are removed by soap and water, or other safe oil-removing products, and gentle drying of the skin is performed prior to its-application. When use of a device 5 is no longer desired, the patient 10 carefully peels it from his or her face 8 and discards it, as said device 5 is intended to be used only once.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A continuous nasal irrigation device for providing moisture to the nasal mucosa of a patient over a prolonged amount of time comprising:
means for holding a fluid;
means for allowing a fluid to flow at a controlled, steady rate through a wick contained in each nostril section of the device to the nasal mucosa of each nostril of the patient;
means for protectively covering said wick;
means for facilitating the transfer of fluid to the nasal mucosa of said patient; and
means for mounting the irrigation device to the face of the patient.

2. The continuous nasal irrigation device in accordance with claim 1 wherein said means for holding a fluid comprises a flexible, contoured bag.

3. The continuous nasal irrigation device in accordance with claim 1, wherein said means for allowing a fluid to flow at a controlled, steady rate through a wick contained in each nostril section of the device to the nasal mucosa of each nostril of the patient comprises a wick, being operatively connected to said bag and having two distinct portions, the exposed wick and the retained wick.

4. The continuous nasal irrigation device in accordance with claim 1, wherein said means for protectively covering said wick comprises a removable cap.

5. The continuous nasal irrigation device in accordance with claim 1, wherein said means for facilitating the transfer of fluid to the nasal mucosa of said patient comprises a flexible, hollow cavity, being disposed in said bag and operatively connected to said retained wick, said cavity containing a fluid.

6. The continuous nasal irrigation device in accordance with claim 1, wherein said means for mounting the irrigation device to the face of the patient comprises an adhesive means, being disposed on said flexible contoured bag.

7. A continuous nasal irrigation device for providing moisture to the nasal mucosa of a patient over a prolonged amount of time comprising:
a flexible, contoured bag, for holding a fluid;
a wick, being operatively connected to said bag and having two distinct portions, the exposed wick and the retained wick, for allowing a fluid to flow at a controlled, steady rate through a wick contained in each nostril section of the device to the nasal mucosa of each nostril of the patient;
a removable cap, for protectively covering said wick;
a flexible, hollow cavity, being disposed in said bag and operatively connected to said retained wick, said cavity containing a fluid, for facilitating the transfer of fluid to the nasal mucosa of said patient; and
an adhesive means, being disposed on said flexible contoured bag, for mounting the irrigation device to the face of the patient.

8. The continuous nasal irrigation device as recited in claim 7, further comprising:
a fluid, for moisturizing the mucosa of the upper respiratory tract, thereby making the mucous less viscous and tenacious while providing the potential for use as a vehicle for delivering medications to the mucosa of the upper respiratory tract, including the nasal cavity, sinuses, and nasopharynx.

9. The continuous nasal irrigation device as recited in claim 7, further comprising:
a nostril section, for frictionally retaining the wick and directing the position of the device into each nostril so that the fluid is transferred to the nasal mucosa.

10. The continuous nasal irrigation device as recited in claim 8, further comprising:
a nostril section, for frictionally retaining the wick and directing the position of the device into each nostril so that the fluid is transferred to the nasal mucosa.

11. The continuous nasal irrigation device as recited in claim 7, wherein said adhesive means is an adhesive means having a peelable adhesive cover.

12. The continuous nasal irrigation device as recited in claim 8, wherein said wick has characteristics selected from the following group: being composed of an absorbent material, and having an adjustable flow rate.

13. The continuous nasal irrigation device as recited in claim 8, wherein said removable cap is a removable cap having a textured grip.

14. The continuous nasal irrigation device as recited in claim 8, wherein said adhesive means is an adhesive means having a peelable adhesive cover.

15. The continuous nasal irrigation device as recited in claim 8, wherein said fluid is liquid, such as physiologic isotonic buffered saline, water, or a solution containing different ingredients or concentrations, including medications.

16. The continuous nasal irrigation device as recited in claim 9, wherein said nostril section is a nostril section having flexible front and back walls allowing for the positioning of the wick in the nostril so that the exposed wick comes in contact with the nasal mucosa.

17. The continuous nasal irrigation device as recited in claim 12, wherein said fluid is liquid, such as physiologic isotonic buffered saline, water, or a solution containing different ingredients or concentrations, including medications.

18. A continuous nasal irrigation device for providing moisture to the nasal mucosa of a patient over a prolonged amount of time comprising:
a flexible, contoured bag, for holding a fluid;
a wick, being operatively connected to said bag and composed of an absorbent material and having two distinct portions, the exposed wick and the retained wick and an adjustable flow rate, for allowing a fluid to flow at a controlled, steady rate through a wick contained in each nostril section of the device to the nasal mucosa of each nostril of the patient;
a removable cap, having a textured grip, for protectively covering said wick;
a flexible, hollow cavity, being disposed in said bag and operatively connected to said retained wick, said cavity containing a fluid, for facilitating the transfer of fluid to the nasal mucosa of said patient;
a liquid, such as physiologic isotonic buffered saline, water, or a solution containing different ingredients or concentrations, including medications fluid, for moisturizing the mucosa of the upper respiratory tract, thereby making the mucous less viscous and tenacious while providing the potential for use as a vehicle for delivering medications to the mucosa of the upper respiratory tract, including the nasal cavity, sinuses, and nasopharynx;
a nostril section, having flexible front and back walls allowing for the positioning of the wick in the nostril so that the exposed wick comes in contact with the nasal mucosa, for frictionally retaining the wick and directing the position of the device into each nostril so that the fluid is transferred to the nasal mucosa; and
an adhesive means, being disposed on said flexible contoured bag and having a peelable adhesive cover, for mounting the irrigation device to the face of the patient.
